(19) United States
(12) Patent Application Publication
(10) Pub. No.: US 2026/0157857 A1
(43) Pub. Date: Jun. 11, 2026

(54) INTERVERTEBRAL DISC IMPLANTS WITH VARYING CUP AND DOME GEOMETRIES

(71) Applicant: CENTINEL SPINE, LLC, West Chester, PA (US)

(72) Inventors: Edward J. McShane, III, Collegeville, PA (US); Timothy J. Bertone, Philadelphia, PA (US); Damian Heinz, West Brandywine Township, PA (US); Thierry Marnay, Montpellier (FR)

(21) Appl. No.: 19/410,279

(22) Filed: Dec. 5, 2025

Related U.S. Application Data
(60) Provisional application No. 63/728,982, filed on Dec. 6, 2024.
(60) Provisional application No. 63/728,974, filed on Dec. 6, 2024.
(60) Provisional application No. 63/728,964, filed on Dec. 6, 2024.

(51) Int. Cl. A61F 2/44; A61F 2/30

(52) U.S. Cl. CPC A61F 2/4425; A61F 2/30749; A61F 2002/30112; A61F 2002/30245; A61F 2002/30326; A61F 2002/30327; A61F 2002/30378; A61F 2002/30621; A61F 2002/443

(57) ABSTRACT

Intervertebral disc implants, including artificial disc replacement implants, are provided that include a first component having an outer surface for engaging one of the adjacent vertebrae and a concave inner surface opposite the outer surface, and a second component having an outer surface for engaging the other of the adjacent vertebrae and an inner surface opposite the outer surface. An articulating core member resides between the first and second components. The articulating core member comprises a convex surface in contact with the concave surface of the first component. The surface area of the convex surface is greater than a surface area of the concave surface, which increases the overall spacing between the endplates. This increased spacing accommodates the deformation that may occur after the implant is inserted in the patient and is subject to load-bearing forces, thereby maintaining disc height and range of motion for a longer period of time.

### CROSS-REFERENCE TO RELATED APPLICATIONS

This application claims the benefit of U.S. Provisional Application Ser. Nos. 63/728,982, 63/728,974 and 63/728,964, all of which were filed on Dec. 6, 2024, the complete disclosures of which are incorporated herein by reference for all purposes.

### TECHNICAL FIELD

Implantable spinal implants, more specifically intervertebral disc implants, and even more specifically artificial disc replacement implants configured to accommodate varying facet and vertebral body orientations and/or loads from the patient's body, are described herein.

### BACKGROUND

Spinal instability is often attributed to undesirable excessive motion between vertebrae which can cause significant pain and morbidity. The instability may result from a number of causes, including abnormalities of the vertebrae, the intervertebral discs, the facet joints, or connective tissue around the spine. These abnormalities may arise from diseases, disorders or defects of the spine from trauma or bone degradation, such as osteoarthritis, or degenerative disc disease. When the spine becomes unstable, the vertebral column becomes misaligned and may allow micromotion between adjacent vertebrae. Vertebral misalignment and micromotion may result in wear to the vertebral bone surfaces and ultimately generate severe pain. These conditions are often chronic and create progressive problems for the sufferer.

Known treatments for spinal instability can include long-term medical management or surgery. Medical management is generally directed at controlling the symptoms, such as pain reduction, rather than correcting the underlying problem. For some patients, this may require chronic use of pain medications, which may alter the patient's mental state or cause other negative side effects. Surgical treatment typically includes decompression procedures to restore normal disc height, realign the column, and alleviate the pain.

Today, a variety of implantable spinal stabilization devices are available to address these spinal conditions or abnormalities in a more permanent manner than with decompression alone. Some of these implantable devices include intervertebral spinal stabilization devices that are configured for placement between adjacent vertebrae. These devices generally fall under the category of either fusion-promoting, or motion-preserving, and may be configured for specific segments of the spine such as the thoracic, lumbar or cervical region of the spine.

One such type of motion-preserving intervertebral spinal stabilization device is the artificial disc implant. Artificial disc implants may be configured as a total artificial disc implant intended to replace the entire disc between two adjacent vertebrae, or a partial artificial disc implant that replaces only a portion while leaving a native remnant of the disc intact. The procedure, known as disc arthroplasty, involves the insertion of an artificial intervertebral disc implant into the intervertebral space between adjacent vertebrae. Such a disc implant allows limited universal movement of the adjacent vertebrae with respect to each other. The aim of total disc replacement is to remove pain generation (caused by a degenerated disc), restore anatomy (disc height), and maintain mobility in the functional spinal unit so that the spine remains in an adapted sagittal balance. Sagittal balance is defined as the equilibrium of the trunk with the legs and pelvis to maintain harmonious sagittal curves and thus the damping effect of the spine. In contrast with fusion techniques, total disc replacement preserves mobility in the motion segment and mimics physiologic conditions.

The biomechanical organization and overall shape of the intervertebral disc space may vary among individual patients. For example, the facet joints may have differing orientations or inclinations that create varying degrees of anatomy within each intervertebral space. In addition, the overall spine may have different curvatures that deviate from the normal curvature of the spine (e.g., lordosis). These varying parameters of individual anatomies may change the rotation mechanics of the disc implant and, in some cases, for example, limit the ability of the implant to rotate in flexion and extension. Thus, it would be desirable to provide disc implants having angled components that can adapt to certain pelvic parameters, such as particular facet joint orientations and varying spinal curvatures.

In use, these artificial disc replacement implants are subject to an enormous amount of anatomical forces. Load-bearing implants that allow motion such as these type of artificial disc implants may experience deformation and shape alterations once placed within the patient where the natural forces such as weight act upon the implant. In particular, the weight of the body may create a load that, over time, creates a deformity in certain components of the endplate, such as the articulating core member of the plate (i.e., the spherical dome that articulates with the upper endplate). This may cause the core member to flatten out such that it has a larger radius of curvature than the concave surface of the upper endplate. In addition, due to subsidence, the upper endplate of these disc implants may settle down and move closer to the lower endplate, which reduces the disc height and its range of motion. Thus, it would also be desirable to provide disc implants that anticipate and accommodate this deformation.

### SUMMARY

Intervertebral disc implants, including artificial disc replacement implants, are provided that are configured for insertion into a disc space disposed between two adjacent vertebral bodies. The implants may be configured for use anywhere along the spine as desired, including the cervical, lumbar or thoracic areas of the spine. These disc implants may be of the type that have an upper endplate, a lower endplate and an articulating core member therebetween that cooperate with one another to provide an articulating joint between the endplates. The implants may be configured to accommodate natural load deformation under the weight of the patient, and can also have angled plates and/or core members to accommodate for different facet joint orientations and varying spinal curvatures, such as lordosis.

In one aspect, an implant for insertion into an intervertebral space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components. The articulating core member comprises a convex surface in contact with the concave surface of the first component. The surface area of the convex surface is greater than the surface area of the concave surface.

In certain embodiments, the first component is an upper or superior endplate and the second component is a lower or inferior endplate. The articulating core member is “oversized” relative to the inner concave surface of the upper endplate, which increases the overall spacing between the endplates. This increased spacing accommodates the deformation that may occur over time with the implant after it is has been implanted into an intervertebral space of a patient, thereby maintaining disc height and range of motion for a longer period of time.

In various embodiments, the ratio between the surface area of the convex surface and the surface area of the concave surface is about 1.5 to about 1.002, or about 1.45 to about 1.05, or about 1.4 to about 1.1, or about 1.35 to about 1.15, or about 1.3 to about 1.2. In an exemplary small cervical disc replacement implant embodiment, the surface area of the convex surface is about 93 mm² to about 110 mm² and the surface area of the concave surface is about 76 mm² to about 93 mm². In an exemplary large cervical disc replacement implant embodiment, the surface area of the convex surface is about 127 mm² to about 144 mm² and the surface area of the concave surface is about 127 mm² to about 110 mm². In an exemplary large lumbar disc replacement implant embodiment, the surface area of the convex surface is about 465 to about 525 mm² and the surface area of the concave surface is about 465 to about 405 mm². In an exemplary small lumbar disc implant replacement embodiment, the surface area of the convex surface is about 410 mm² to about 466 mm² and the surface area of the concave surface is about 353 mm² to about 409 mm². These surface areas are calculated using the geometry equation for partial spherical surface area [surface area (SA) equals 2×pi×radius×height].

In some embodiments, the spherical radius of the convex surface of the articulating core member is greater than the spherical radius of the concave surface of the first component. In an exemplary embodiment, the ratio between spherical radius of the convex surface and the spherical radius of the concave surface is about 1.5 to about 1.002, or about 1.45 to about 1.05, or about 1.4 to about 1.1, or about 1.35 to about 1.15, or about 1.3 to about 1.2.

In various embodiments, the upper endplate is configured to articulate in the posterior or anterior direction (i.e., around a lateral axis generally perpendicular to a longitudinal or posterior-anterior axis of the endplate) relative to the lower endplate from a first anatomically neutral position, wherein the upper and lower endplates are substantially parallel, to a second position, wherein the upper endplate is disposed at an angle of at least about 7 degrees, or at least 11.5 degrees relative to the lower endplate.

In other embodiments, the upper endplate also has an increased range of motion in the lateral direction (i.e., rotating around an axis parallel to the longitudinal axis of the implant). In certain embodiments, the upper endplate is configured to articulate in the lateral direction relative to the lower endplate from a first anatomically neutral position, wherein the upper and lower endplates are substantially parallel, to a second lateral position, wherein the upper endplate 110 is disposed at an angle of at least about 7 degrees, or at least 11.5 degrees relative to lower endplate 120.

In various embodiments, the articulating core member comprises a semi-spherical member and an inlay. The inlay may be coupled to the lower implant and comprises a recess for receiving the semi-spherical member. Alternatively, the inlay and the semi-spherical member may be formed from a single integral component.

In various embodiments, the upper and lower endplates each comprise a fixation element extending from their outer surfaces. In one such embodiment, the fixation element(s) comprise one or more spikes. In other embodiments, the fixation elements comprises one or more keels. In yet another embodiment, the fixation elements comprise a combination of spikes and keels.

In various embodiments, the intervertebral disc replacement prosthesis is sized to fit a cervical and cervico-thoracic intervertebral disc space (C2-T1). In other various embodiments, the intervertebral disc replacement prosthesis is sized to fit the lumbar, thoracolumbar, or lumbosacral (T12-S1) disc spaces, predominantly the human disc spaces from L2 to S1 vertebrae.

In another aspect, an implant for insertion into an intervertebral space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components. The articulating core member comprises a convex surface in contact with the concave surface of the first component. The convex surface defines an arc from one end of the convex surface to an opposite end of the convex surface and the concave surface defines an arc from one end of the concave surface to an opposite end of the concave surface. The length of the convex surface arc is greater than the length of the concave surface arc.

In various embodiments, the ratio of the length of the cup's convex surface arc radius to the length of the concave surface arc radius is about 1.5 to about 1.002, or about 1.45 to about 1.05, or about 1.4 to about 1.1, or about 1.35 to about 1.15, or about 1.3 to about 1.2. The length of the cup's convex surface arc radius is about 5.51 mm to about 6.5 mm for smaller cervical prostheses and increased to about 7.51 mm to about 8.5 mm for larger cervical prostheses. The dome's mating length radii of the concave surface is about 5.50 mm to about 4.5 mm for smaller cervical prostheses, and about 7.50 to about 6.5 mm for larger cervical prostheses.

In various embodiments for larger parts of the human spine, mostly lumbar and lumbosacral spine, the length of the cup's convex surface arc radius is about 14.51 mm to about 16.5 mm for smaller lumbar prostheses and increased to about 16.51 mm to about 18.5 mm for larger cervical prostheses. The dome's mating length radii of the concave surface is about 14.50 mm to about 12.5 mm for smaller lumbar prostheses, and about 16.50 mm to about 14.5 mm for larger lumbar prostheses.

In another aspect, an implant for insertion into an intervertebral space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components. The articulating core member comprises a semi-spherical protrusion with a convex surface in contact with the concave surface of the first component. The radius of curvature of the convex surface of the core member is different from the radius of curvature of the concave surface of the first component.

In various embodiments, the first component is an upper or superior endplate and the second component is a lower or inferior endplate. The radius of curvature of the concave surface of the upper endplate is greater than the radius of curvature of the convex surface of the semi-spherical protrusion or dome. In other words, the convex and concave surfaces each generally define an arc of a circle having a certain radius. The radius of the circle defined by the concave surface arc of the upper endplate is greater than the radius of the circle defined by the convex surface arc of the dome. This allows the upper endplate to adapt to the shape of the spherical dome under a load. For example, when the spherical dome is under a load from the patient's body, it will deform slightly into a flatter or larger radius of curvature (i.e., compress and flatten out). Providing an upper endplate with a larger radius of curvature at the outset (i.e., before implanting the implant and before the implant is under the load of the body) allows the upper endplate to adapt to the deformation of the spherical dome under load.

In various embodiments, the radius of the concave surface of the upper endplate is selected to be substantially equal to the deformed radius of the spherical dome under load. This load may depend on a variety of factors, such as the location of the implant in the patient's spine (i.e., lumbar implants may be subject to greater loads than cervical implants), the overall weight of the patient, and other anatomical factors. In an exemplary embodiment, the radius of the concave surface is about 10 microns to about 100 microns greater than the radius of the convex surface of the spherical dome.

In another aspect, an implant for insertion into an intervertebral space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components. The articulating core member comprises a semi-spherical protrusion with a convex surface in contact with the concave surface of the first component. The semi-spherical protrusion is disposed at a transverse angle relative to the inner surface of the second component.

In various embodiments, the semi-spherical protrusion is titled towards the posterior or leading direction of the implant. This creates an implant with a built-in lordotic angle that may accommodate or compensate for a natural degree of posterior curvature within a portion of the patient's spine (i.e., lumbar or cervical lordosis).

In various embodiments, the inner surface of the second component extends along a plane substantially parallel to a longitudinal axis of the implant. The semi-spherical protrusion defines a center of curvature and an axis extending from the center of curvature to an outer surface of the protrusion. The axis extends at an acute angle relative to the inner surface of the second component such that the semi-spherical protrusion is “tilted” relative to the second component. In an exemplary embodiment, the acute angle is about 75 degrees to about 89 degrees, or about 85 degrees to about 88 degrees, or about 87 degrees.

In various embodiments, the semi-spherical protrusion comprises a first semi-spherical surface and a second surface extending from the semi-spherical surface to the inner surface of the second component. The second surface may be transverse to, or substantially perpendicular to, the inner surface of the second component. At least a portion of the first semi-spherical surface may contact at least a portion of the inner surface of the second component.

In various embodiments, the semi-spherical protrusion is positioned posteriorly to the center of the first and/or second components. In one such embodiment, the second component has a posterior end and an anterior end and a midline axis extending through the second component equally distant between the anterior and posterior ends. The semi-spherical protrusion comprises a dome having an apex that is posterior to the midline axis of the second component. Thus, the implant is designed to accommodate a vertebral joint (or functional spinal unit) with a center of rotation naturally located more posteriorly, for example, when one or more of the facets are more inclined towards a vertical orientation than normal

In various embodiments, the first component comprises an upper endplate and the second component comprises a lower endplate. The apex of the dome is posteriorly spaced about 2 mm to about 10 mm, or about 2 mm to about 10 mm from the midline axis of the lower endplate. The apex of the dome may be spaced about 13 mm to about 6 mm from a posterior surface of the lower endplate. Thus, the center of rotation (COR) is approximately 50-80% of the plate A-P depth of the inferior plate matched to the overall depth of the vertebral endplate.

In various embodiments, the convex surface of the dome defines a posterior surface area located posterior to the midline axis of the lower endplate and an anterior surface area located anterior to the midline axis of the lower endplate. The posterior surface area is greater than the anterior surface area. In an exemplary embodiment, the posterior surface area is about 45% to about 80%, or about 50% to about 75%, or about 55% to about 70%, of the anterior surface area.

In various embodiments, the articulating core member comprises a semi-spherical member and an inlay. The inlay may be coupled to the second component and comprises a recess for receiving the semi-spherical member. Alternatively, the inlay and the semi-spherical member may be formed from a single integral component.

In various embodiments, the first component is an upper or superior endplate and the second component is a lower or inferior endplate. The upper and lower endplates may each comprise a fixation element extending from their outer surfaces. In one such embodiment, the fixation element(s) comprise one or more spikes. In other embodiments, the fixation elements comprise one or more keels. In yet another embodiment, the fixation elements comprise a combination of spikes and keels.

In various embodiments, the intervertebral disc space is a cervical intervertebral disc space.

In another aspect, an implant for insertion into an intervertebral space between two adjacent vertebrae comprises a first component having an outer surface for engaging one of the adjacent vertebrae and an internal cup having an internal concave surface with a substantially annular base surface. The first component further comprises an inner surface substantially surrounding the concave surface opposite the outer surface. The implant further comprises a second component having an outer surface for engaging the other of the adjacent vertebrae and an inner surface opposite the outer surface and an articulating core member residing between the first and second components. The articulating core member comprises a convex surface in contact with the concave surface of the internal cup of the first component. The base surface of the internal cup is disposed at a transverse angle relative to the inner surface of the first component.

In an exemplary embodiment, this transverse angle is about 1 degree to about 15 degrees or about 5 degrees to about 2 degrees or about 3 degrees.

In various embodiments, the first component is an upper superior endplate and the second component is a lower inferior endplate and at least a portion of the internal cup is disposed between the inner surfaces of the upper and lower endplates. Thus, the lower surface of the upper endplate is “tilted” towards the posterior or leading direction of the implant relative to the internal cup. This creates an implant with a built-in lordotic angle that may accommodate or compensate for a natural degree of posterior curvature within a curved portion of the patient's spine (i.e., cervical or lumbar lordosis).

This implant is particularly useful in a disc space that is in extension (i.e., space lordosis) such that the upper or superior vertebrae is angled relative to the lower vertebrae. In this type of disc space, the upper endplate resides at an angle relative to the lower endplate in the anatomically neutral position, which generally reduces the amount of room left in the space for the upper endplate to rotate relative to the lower endplate. In this embodiment, the angled superior endplate absorbs at least a part of the requirement for a lordosis angle and increases the range of motion in extension instead of using this degree of angle in static compensation.

In various embodiments, the internal cup defines a first side surface and a second side surface extending from the inner surface of the upper endplate. The length of the first side surface is greater than a length of the second side surface. In an exemplary embodiment, the first side surface is an anterior side surface and the second side surface is a posterior side surface.

In various embodiments, the upper endplate is movable between a first anatomically neutral position to a second rotated position. In the first anatomically neutral position, the distal base surface of the internal cup is substantially parallel to the inner surface of the lower endplate and the inner surface of the upper endplate is disposed at a transverse angle relative to the inner surface of the lower endplate. In an exemplary embodiment, this transverse angle is about 1 degree to about 15 degrees, or about 5 degrees to about 2 degrees, or about 3 degrees.

In various embodiments, the upper and lower endplates may each comprise a fixation element extending from their outer surfaces. In one such embodiment, the fixation element(s) comprise one or more spikes. In other embodiments, the fixation elements comprise one or more keels. In yet another embodiment, the fixation elements comprise a combination of spikes and keels.

In another aspect, an implant for insertion into an intervertebral space between two adjacent vertebrae comprises a first component having an outer surface for engaging one of the adjacent vertebrae and a concave surface opposite the outer surface. The first component further comprises an inner surface substantially surrounding the concave surface. The implant further comprises a second component having an outer surface for engaging the other of the adjacent vertebrae and an inner surface opposite the outer surface, and an articulating core member residing between the first and second components. The articulating core member comprising a convex surface in contact with the concave surface of the first component. The inner surface of the second component is disposed at a transverse angle relative to the inner surface of the first component.

In various embodiments, the first component is an upper superior endplate and the second component is a lower inferior endplate and at least a portion of the internal cup is disposed between the inner surfaces of the upper and lower endplates. This creates an implant with a built-in lordotic angle that may accommodate or compensate for a natural degree of posterior curvature within a curved portion of the patient's spine (i.e., cervical or lumbar lordosis).

This implant is particularly useful in a disc space wherein the anatomical shape of the vertebra is trapezoidal such that the anterior portion is higher than the posterior portion. The angled lower endplate reduces the angle between the facet and the superior endplate, which increases the translation or range of motion in flexion. In addition, the configuration may horizontalize the joint and reduce shear forces during rotation of the implant.

In various embodiments, the articulating core member comprises a dome having an apex and a radius extending downwardly from the apex towards a center of the core member. The inner surface of the second component is disposed at a transverse angle relative to the radius. In an exemplary embodiment, this transverse angle is about 75 degrees to about 89 degrees, or about 85 degrees to about 88 degrees or about 87 degrees.

In various embodiments, the upper endplate is movable between a first anatomically neutral position to a second rotated position. In the first position, the inner surface of the upper endplate is substantially parallel to the radius and the inner surface of the lower endplate is disposed at a transverse angle relative to the radius.

In various embodiments, the upper and lower endplates may each comprise a fixation element extending from their outer surfaces. In one such embodiment, the fixation element(s) comprise one or more spikes. In other embodiments, the fixation elements comprise one or more keels. In yet another embodiment, the fixation elements comprise a combination of spikes and keels.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A is a top view of a spinal implant;

FIG. 1B is a cross-sectional view of a spinal implant taken from a section along axis A-A of FIG. 1A;

FIG. 1C is a cross-sectional view of the spinal implant of FIG. 1A taken from a section along axis B-B of FIG. 1A;

FIG. 2A is an exploded view of an articulating core and an inlay of the spinal implant of FIGS. 1A-1C;

FIG. 2B is a cross-sectional view of the articulating core and the inlay of FIG. 2A;

FIG. 3A illustrates the spinal implant of FIGS. 1A-1C in an anatomically neutral position;

FIG. 3B illustrates the spinal implant of FIGS. 1A-1C in a posteriorly rotated position;

FIG. 4A is a cross-sectional view of another spinal implant taken from a section along the longitudinal axis of the implant;

FIG. 4B is a cross-sectional view of another spinal implant taken from a section along a lateral axis of the implant;

FIG. 5A is a cross-sectional view of another spinal implant taken from a section along the longitudinal axis of the implant (section A-A in FIG. 5C);

FIG. 5B is a cross-sectional view of the spinal implant of FIG. 5A taken from a section along a lateral axis of the implant (section B-B in FIG. 5C);

FIG. 5C is a top view of the spinal implant of FIGS. 5A and 5B;

FIG. 6A schematically illustrates a portion of another spinal implant with no load applied to the implant;

FIG. 6B schematically illustrates the spinal implant of FIG. 6A, illustrating translation of the upper endplate relative to the articulating core;

FIG. 6C schematically illustrates the spinal implant of FIG. 6A, illustrating rotation of the upper endplate relative to the articulating core;

FIG. 6D schematically illustrates the spinal implant of FIG. 6A with a load applied to the implant;

FIG. 7A is a perspective view of another spinal implant;

FIG. 7B illustrates an articulating core and an inlay of the spinal implant of FIG. 7A;

FIG. 8A is a perspective view of another spinal implant;

FIG. 8B illustrates an articulating core and an inlay of the spinal implant of FIG. 8A;

FIG. 8C schematically illustrates a prior art spinal implant in an intervertebral space with lordosis;

FIG. 8D schematically illustrates the spinal implant of FIGS. 8A and 8B in an intervertebral space with lordosis;

FIG. 9A is a side view of another spinal implant;

FIG. 9B schematically illustrates a prior art spinal implant in an intervertebral space having a trapezoidal shape with an anterior portion higher than a posterior portion; and

FIG. 9C schematically illustrates the spinal implant of FIG. 9A in an intervertebral space having a trapezoidal shape with an anterior portion higher than a posterior portion.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a”, “an”, and “the”, and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Spinal devices are provided that are configured for implantation within an intervertebral space disposed between vertebral bodies. The vertebral bodies can be anatomically adjacent vertebral bodies, or can be the vertebral bodies that remain after a discectomy has been performed that removed a vertebral body from a location between the vertebral bodies. The devices are configured to be inserted into the intervertebral space, and achieve restoration of height while maintaining mobility. The intervertebral space can be disposed anywhere along the spine as desired, including the cervical, lumbar or thoracic areas of the spine. The implants described herein are particularly useful for accommodating the deformation that may occur over time with the implant after it is has been implanted into an intervertebral space of a patient, thereby maintaining disc height and freedom of motion for a longer period of time. In some embodiments, the implants described herein include a built-in lordotic angle that may accommodate a degree of posterior curvature within a curved portion of the patient's spine.

Certain terminology is used in the following description for convenience only and is not limiting. The words “right”, “left”, “lower” and “upper” designate directions in the drawings to which reference is made. The words “inner” or “distal” and “outer” or “proximal” refer to directions toward and away from, respectively, the geometric center of the implant and related parts thereof. The words “anterior”, “posterior”, “superior”, “inferior”, “medial”, “lateral”, and related words and/or phrases designate preferred positions and orientations in the human body to which reference is made and are not meant to be limiting. The terminology includes the above listed words, derivatives thereof and words of similar import.

Unless otherwise specified herein, the terms “lateral”, “longitudinal”, and “transverse” are used to describe the orthogonal directional components of various components. It should be appreciated that while the longitudinal and lateral directions are illustrated as extending along a horizontal plane, and that the transverse direction is illustrated as extending along a vertical plane, the planes that encompass the various directions may differ during use. For instance, when the implant is implanted into an intervertebral space, such as the intervertebral space, the transverse direction extends generally along the superior-inferior (or caudal-cranial) direction, while the plane defined by the longitudinal direction and lateral directions generally in the anatomical plane defined by the anterior-posterior direction, and the medial-lateral direction. Accordingly, the directional terms “vertical” and “horizontal” are used to describe the implants and their components as illustrated merely for the purposes of clarity and illustration.

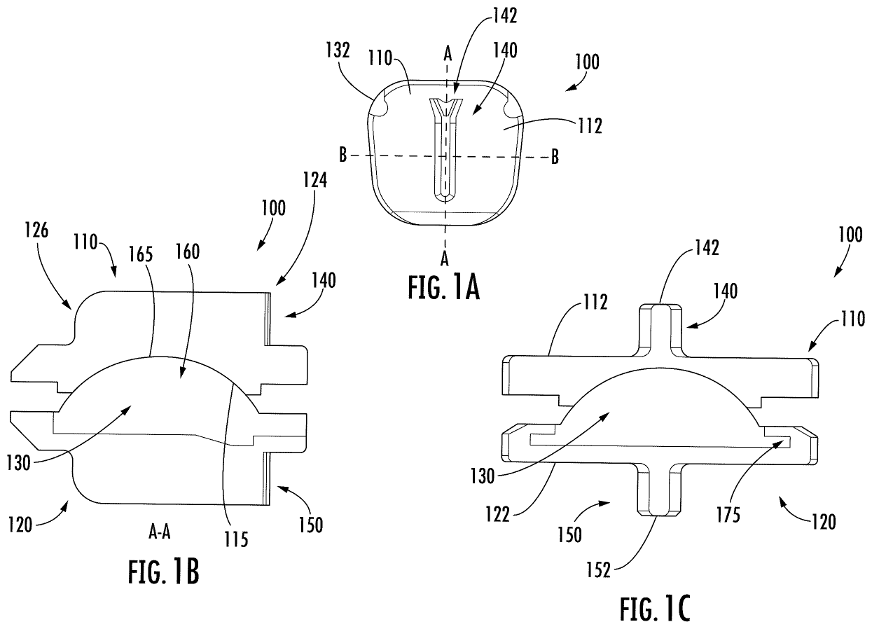

For purposes of clarity, the longitudinal axis is hereinafter defined as the axis generally extending from an anterior portion of the implant to a posterior portion of the prosthesis after insertion of the implant into the intervertebral body (axis A-A in FIG. 1A). The anterior portion may also be considered the trailing end of the prosthesis and the posterior portion the leading end (with reference to the direction the prosthesis is introduced into the intervertebral space). The lateral axis is hereafter defined as the axis perpendicular to the longitudinal axis and generally extending from one lateral side of the implant to the other lateral side (axis B-B in FIG. 1A).

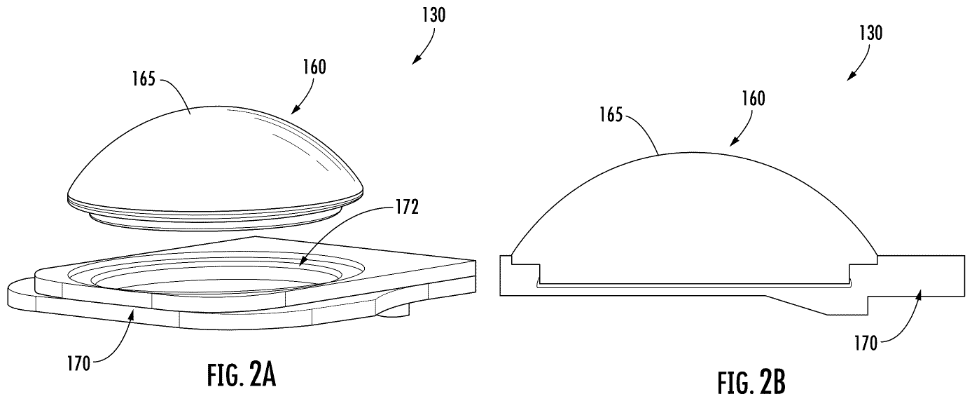

Referring now to FIGS. 1A-1C, 2A and 2B, a spinal implant 100 comprises an upper or superior endplate 110, a lower or inferior endplate 120 and a core 130. Superior endplate 110 rides upon the core 130 and is operable to rotate relative to core 130, including torsional rotation about a vertical axis, flexion/extension rotation in the posterior/anterior directions and lateral bending rotation. Core 130 includes a substantially spherical dome 160 having a convex upper surface 165 that contacts an internal concave surface 115 of upper endplate 110 and an inlay 170 that couples dome 160 to lower endplate 110. The spherical dome 160 may be a separate element from the inlay 170 and coupled thereto, or the core and inlay may be manufactured as a single, integral element.

Core 130 may be coupled to lower endplate 110 in any suitable manner. In an exemplary embodiment, dome 160 is coupled to an upper recess 172 within inlay 170, and inlay 170 is slidably disposed within longitudinal recesses or slots 175 in lower endplate 110. For example, inlay 170 may include a plastic snap-in projection that fits within snap-in recesses or slots 175 in lower endplate 120 such that the plastic inlay can snap into place but is thereafter inhibited from being removed.

Convex upper surface 165 of dome 160 has a surface area that is larger than the surface area of concave inner surface 115 of upper endplate 110. Accordingly, dome 160 is “oversized” relative to concave inner surface 115, which increases the overall spacing between upper and lower endplates 110, 120. This increased spacing accommodates the deformation that occurs over time with implant 110 after it is has been implanted into an intervertebral space of a patient. In other words, the oversized dome compensates for the natural wear and shape change that typically occurs with the implant to maintain disc height and freedom of motion for a longer period of time.

In some embodiments, the ratio between the surface area of the convex surface and the surface area of the concave surface is about 1.5 to about 1.002, or about 1.45 to about 1.05, or about 1.4 to about 1.1, or about 1.35 to about 1.15, or about 1.3 to about 1.2. In an exemplary small cervical disc replacement implant embodiment, the surface area of the convex surface is about 93 mm² to about 110 mm² and the surface area of the concave surface is about 76 mm² to about 93 mm². In an exemplary large cervical disc replacement implant embodiment, the surface area of the convex surface is about 127 mm² to about 144 mm² and the surface area of the concave surface is about 127 mm² to about 110 mm². In an exemplary large lumbar disc replacement implant embodiment, the surface area of the convex surface is about 465 mm² to about 525 mm² and the surface area of the concave surface is about 465 mm² to about 405 mm². In an exemplary small lumbar disc replacement implant embodiment, the surface area of the convex surface is about 410 mm² to about 466 mm² and the surface area of the concave surface is about 353 mm² to about 409 mm². These surface areas are calculated using the geometry equation for partial spherical surface area [surface area (SA) equals 2×pi×radius×height].

The following table provides additional details for various embodiments of small and large cervical disc replacement implants, and small and large lumbar disc replacement implants, consistent with the ranges noted above.

Equation Surface Area equals 2 × pi × r × h

| | Small | Surface Area | Percentage | Large | Surface Area | Percentage |
|---|---|---|---|---|---|---|
| | Cervical | Sq. mm | of Nom. | Cervical | Sq. mm | of Nom. |
| Min. OverSized Cup | SR5.51 | 93.4749492 | 100.2% | SR7.51 | 127.4041504 | 100.1% |
| Max. OverSized Cup | SR6.5 | 110.2699038 | 118.2% | SR8.5 | 144.1991049 | 113.3% |
| Nominal Dome | SR5.5 | 93.30630319 | 100.0% | SR7.5 | 127.2345044 | 100.0% |
| Min. UnderSized Dome | SR4.5 | 76.34070261 | 81.8% | SR6.5 | 110.2699038 | 86.7% |

| | Small | Surface Area | Percentage | Large | Surface Area | Percentage |
|---|---|---|---|---|---|---|
| | Lumbar | Sq. mm | of Nom. | Lumbar | Sq. mm | of Nom. |
| Min. OverSized Cup | SR14.51 | 410.2605907 | 100.1% | SR16.51 | 466.8092593 | 100.1% |
| Max. OverSized Cup | SR16.5 | 466.526516 | 113.8% | SR18.5 | 523.0751846 | 112.1% |
| Nominal Dome | SR14.5 | 409.9778474 | 100.0% | SR16.5 | 466.526516 | 100.0% |
| Min. UnderSized Dome | SR12.5 | 353.4291788 | 86.2% | SR14.5 | 409.9778474 | 87.9% |

In some embodiments, the spherical radius of the convex surface of the articulating core member is greater than the spherical radius of the concave surface of the first component. In an exemplary embodiment, the ratio between spherical radius of the convex surface and the spherical radius of the concave surface is about 1.5 to about 1.002, or about 1.45 to about 1.05, or about 1.4 to about 1.1, or about 1.35 to about 1.15, or about 1.3 to about 1.2.

Convex surface 165 defines an arc from one end of the convex surface to an opposite end. Similarly, concave surface 115 defines an arc from one end of the concave surface to an opposite end. In various embodiments, the ratio of the length of the cup's convex surface arc radius to the length of the concave surface arc radius is about 1.5 to about 1.002, or about 1.45 to about 1.05, or about 1.4 to about 1.1, or about 1.35 to about 1.15, or about 1.3 to about 1.2. The length of the cup's convex surface arc radius is about 5.51 mm to about 6.5 mm for smaller cervical prostheses and increased to about 7.51 mm to about 8.5 mm for larger cervical prostheses. The dome's mating length radii of the concave surface is about 5.50 mm to about 4.5 mm for smaller cervical prostheses, and about 7.50 mm to about 6.5 mm for larger cervical prostheses.

In various embodiments for larger parts of the human spine, mostly lumbar and lumbosacral spine, the length of the cup's convex surface arc radius is about 14.51 mm to about 16.5 mm for smaller lumbar prostheses and increased to about 16.51 mm to about 18.5 mm for larger cervical prostheses. The dome's mating length radii of the concave surface is about 14.50 mm to about 12.5 mm for smaller lumbar prostheses, and about 16.50 mm to about 14.5 mm for larger lumbar prostheses.

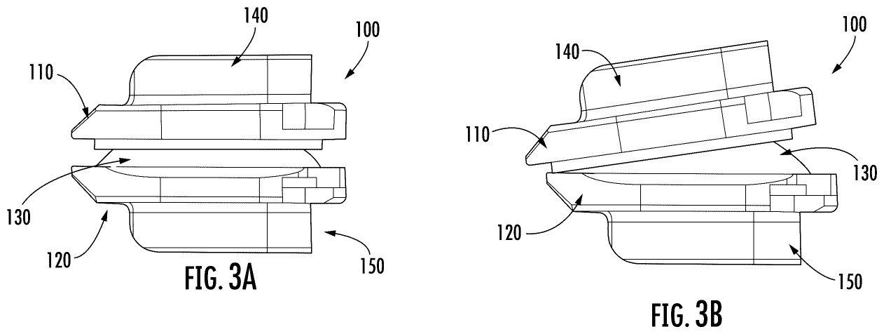

The oversized dome configuration also provides an increased range of motion of implant 100. In particular, since upper endplate 110 is spaced further away from lower endplate 120 and convex surface 165 of dome 160 has a larger surface area than inner concave surface 115 of upper endplate 110, upper endplate 110 may rotate relative to lower endplate 120 over a greater total angle. As shown in FIGS. 3A and 3B, upper endplate 110 is configured to articulate in the posterior or anterior direction (i.e., around axis B-B shown in FIG. 1A) relative to lower endplate 120 from a first anatomically neutral position (FIG. 3A), wherein upper and lower endplates 110, 120 are substantially parallel, to a second position (FIG. 3B), wherein the upper endplate 110 is disposed at an angle of at least about 10 degrees, or at least 11.5 degrees relative to lower endplate 120.

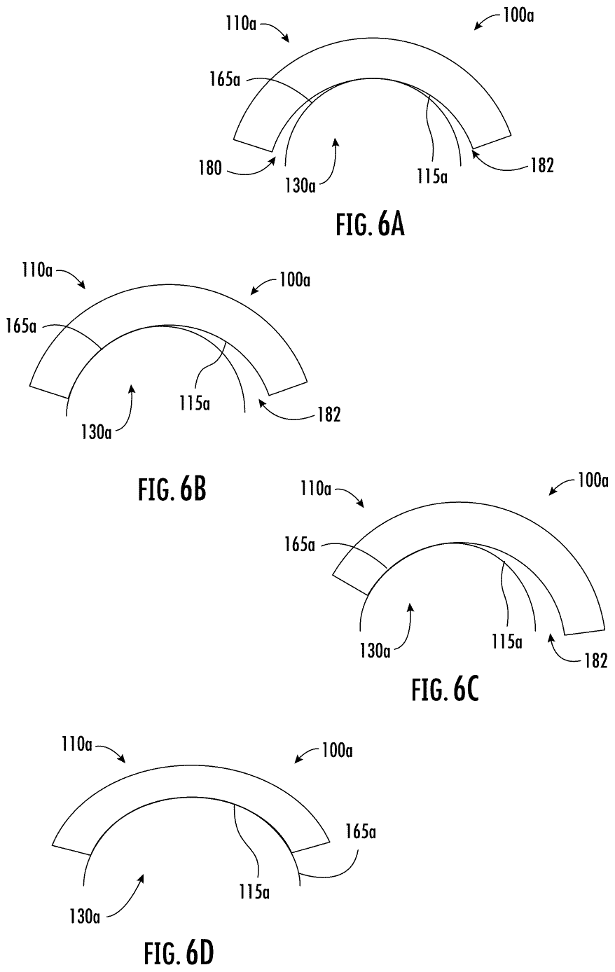

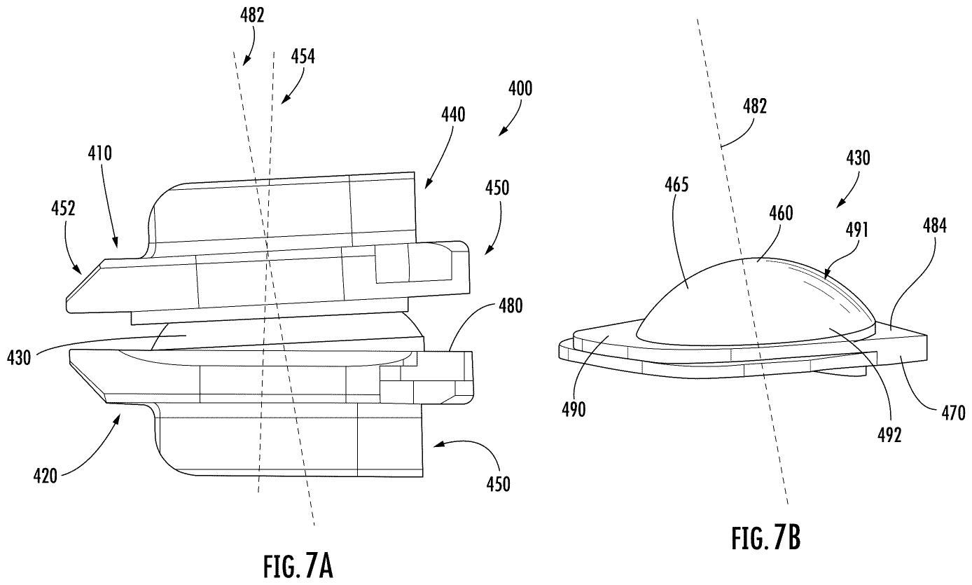

In certain embodiments, upper surface 165 of dome 160 has substantially the same radius of curvature as lower convex surface 115 of endplate 100. In other embodiments, upper surface 165 may have a larger radius of curvature (i.e., a flatter surface) to accommodate a deformation in dome 160 under load that reduces its radius of curvature (see, for example, the embodiment described below in reference to FIGS. 6A and 6B).

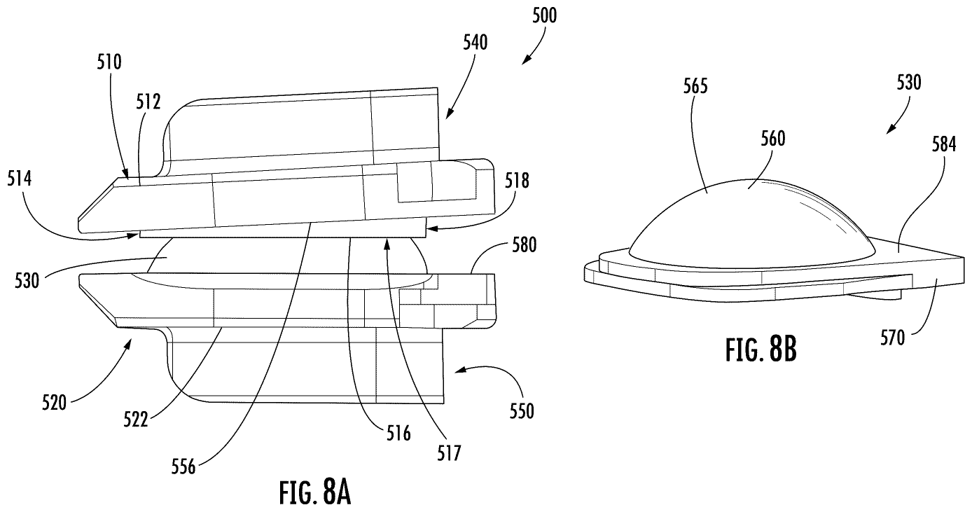

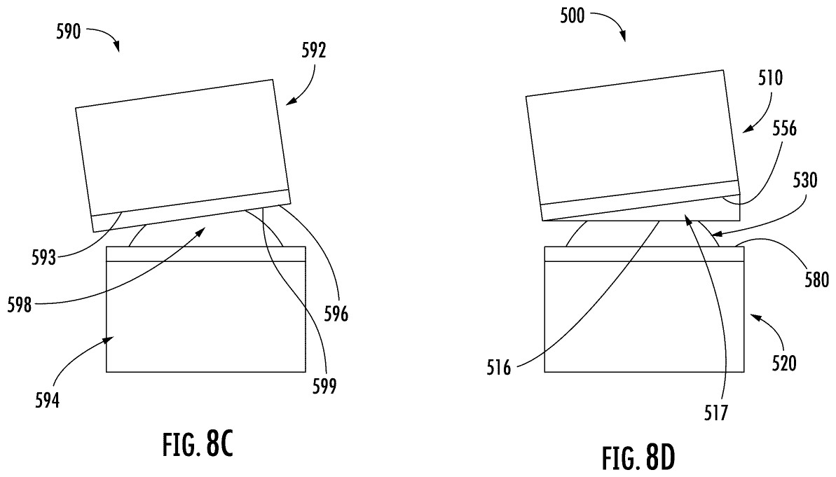

In certain embodiments, convex surface 115 may be part of a separate internal cup that is coupled to upper endplate 110. In these embodiments, upper endplate 110 may have a lower surface that extends around the internal cup with the cup extending downward from this lower surface so that at least part of the internal cup is disposed between the endplates (see, for example, the embodiments described below in reference to FIGS. 8A and 8B).

Upper endplate 110 may also have an increased range of motion in the lateral direction (i.e., rotating around axis A-A in FIG. 1A). In certain embodiments, upper endplate 110 is configured to articulate in the lateral direction relative to lower endplate 120 from a first anatomically neutral position (FIG. 3A), wherein upper and lower endplates 110, 120 are substantially parallel, to a second lateral position (not shown), wherein the upper endplate 110 is disposed at an angle of at least about 7 degrees, or at least 11.5 degrees relative to lower endplate 120.

In one embodiment, the upper bone contacting surface 112 of upper endplate 110 is substantially flat and the side surfaces of upper endplate 110 are substantially perpendicular to the upper surface and include rounded or beveled corners. Similarly, the lower bone contacting 122 surface of lower endplate 120 is substantially flat and the side surfaces of lower endplate 120 are substantially perpendicular to the lower surface and include rounded or beveled corners. In some embodiments, upper and/or lower endplates 110, 120 have recesses or notches 132 carved into the two anterior corners.

In other embodiments, bone contacting surface 112 (and/or surface 122) may have a substantially domed shape such that part of, or the entire, contact surface is curved. In some embodiments, the contact surfaces may be generally convex from an anterior edge of each surface to the posterior edge of each surface. Similarly, the contact surfaces may be generally convex from a first lateral edge of the surface to a second lateral edge opposite the first lateral edge.

Implant 100 further includes a first keel 140 extending from upper bone contacting surface 112 of upper endplate 110 and a second keel 150 extending from lower bone contacting surface 122 of lower endplate 120. Keels 120, 140 are centrally located on endplates 110, 120 such that they generally extend along a longitudinal axis of the endplates.

In certain embodiments, keel 140 may include a recess 142 that opens upwardly and anteriorly and keel 150 includes a recess 152 that opens downwardly and anteriorly. Recesses 142, 152 extend from a trailing end 124 of keels 140, 150 to a portion of keels 140, 150 located between the trailing and leading ends 124, 126 of keels 140, 150, preferably about 25% to about 75% of the distance between the leading and trailing ends, or about 50%. The recesses 142, 152 extend from the bone contacting surfaces 112, 122 of endplates 110, 120 to the upper surface of keels 140, 150 and generally have a width of about 20% to about 50% of the overall width of the keels.

The trailing end 124 and the side surfaces of keels 140, 150 are substantially perpendicular to the bone contacting surfaces of endplates 110, 120. The upper surface of keels 140, 150 are substantially parallel to the bone contacting surfaces of endplates 110, 120. The leading ends 126 of the keels 140, 150 are rounded or beveled to facilitate insertion of the keels into cutouts formed in the adjacent vertebrae. A more complete description of suitable keels that may be incorporated into implant 100 can be found in commonly assigned, U.S. Pat. No. 7,204,852, the complete disclosure of which is incorporated herein by reference for all purposes.

Upper and lower endplates 110, 120 and components thereof, can be formed of any suitable biocompatible material, such as cobalt chromium molybdenum (CoCrMo), titanium and titanium alloys, stainless steel, ceramics, or polymers such as polyetheretherketone (PEEK), polyetherketoneketone (PEKK), bioresorbable materials, and bone graft (for example, allografts and xenografts). A coating may be added or applied to the endplates to improve physical or chemical properties. The coatings may help to ensure bony in or on growth or medication. Examples of coatings include plasma-sprayed titanium coating or hydroxyapatite. The porosity of the coating ideally permits vascularization and osteoblast formation with subsequent bony on-growth.

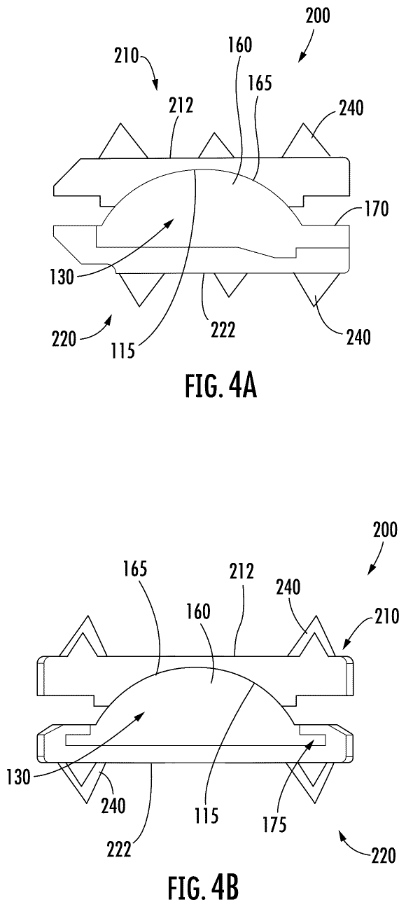

Referring now to FIGS. 4A and 4B, another embodiment of a spinal implant 200 comprises an upper or superior endplate 210, a lower or inferior endplate 220 and a core 130. Superior endplate 210 rides upon the core 130 and is operable to rotate relative to core 130, including torsional rotation about a vertical axis, flexion/extension rotation in the posterior/anterior directions and lateral bending rotation. Similar to the previous embodiment, core 130 includes a substantially spherical dome 160 having a convex upper surface 165 that contacts an internal concave surface 115 of upper endplate 210 and an inlay 170 that couples dome 160 to lower endplate 220. In an exemplary embodiment, inlay 170 is slidably disposed within longitudinal recesses or slots 175 in lower endplate 220. For example, inlay 170 may include a plastic snap-in projection that fits within snap-in recesses or slots 175 in lower endplate 120 such that the plastic inlay can snap into place but is thereafter inhibited from being removed. The spherical dome 160 may be a separate element from the inlay 170 and coupled thereto, or the core and inlay may be manufactured as a single, integral element.

Convex upper surface 165 of dome 160 has a surface area that is larger than the surface area of concave inner surface 115 of upper endplate 210. Accordingly, dome 160 is “oversized” relative to concave inner surface 115, which increases the spacing between upper and lower endplates 210, 220 in the anatomically neutral position shown, for example, in FIG. 4A. In various embodiments, the ratio between the surface area of the convex surface 165 and the surface area of the concave surface 115 is about 1.5 to about 1.002, or about 1.45 to about 1.05, or about 1.4 to about 1.1, or about 1.35 to about 1.15, or about 1.3 to about 1.2 In some embodiments, the spherical radius of the convex surface 165 of dome 160 is greater than the spherical radius of the concave surface 115 of upper endplate 210. In an exemplary embodiment, the ratio between spherical radius of the convex surface and the spherical radius of the concave surface is about 1.5 to about 1.002, or about 1.45 to about 1.05, or about 1.4 to about 1.1, or about 1.35 to about 1.15, or about 1.3 to about 1.2.

Also similar to the previous embodiments, the oversized dome configuration also provides an increased range of motion of implant 100. In particular, since upper endplate 210 is spaced further away from lower endplate 220 and dome 160 has a larger surface area than inner concave surface 115 of upper endplate 210, upper endplate 210 may rotate relative to lower endplate 220 over a greater total angle about axes A-A and/or B-B (shown in FIG. 1).

In one embodiment, the upper bone contacting surface 212 of upper endplate 210 is substantially flat and the side surfaces of upper endplate 210 are substantially perpendicular to the upper surface and include rounded or beveled corners. Similarly, the lower bone contacting 222 surface of lower endplate 220 may be substantially flat and the side surfaces of lower endplate 220 are substantially perpendicular to the lower surface and include rounded or beveled corners. In some embodiments, upper and/or lower endplates 210, 220 have recesses or notches (not shown) carved into one or more of the corners.

In other embodiments, bone contacting surfaces 212 and/or surface 222 may have a substantially domed shape such that part of, or the entire, contact surface is curved. In some embodiments, the contact surfaces may be generally convex from an anterior edge of each surface to the posterior edge of each surface. Similarly, the contact surfaces may be generally convex from a first lateral edge of the surface to a second lateral edge opposite the first lateral edge.

Upper and lower endplates 210, 240 each include at least one spike 240, such as a plurality of spikes, projecting from the bone facing surfaces 212, 222 of the endplate bodies. The spikes may be arranged in first and second substantially symmetrical and substantially identically constructed groups. In certain embodiments, one group of spikes is disposed in a first lateral region of the endplate body, and the second group of spikes is disposed in a second lateral region of the endplate body opposite the longitudinal axis from the first group of spikes. Each lateral group includes a first or longitudinally forward or front spike, a second or longitudinally middle spike, and a third or longitudinally rear spike, such that the longitudinally middle spike is disposed longitudinally between the forward spike and the rear spike, and forward of the central lateral axis. The spikes of each lateral group can be constructed substantially identically and symmetrically. In some embodiments, the spikes in each group are laterally spaced from each other relative to the longitudinal axis.

In other embodiments, the spikes may have different configurations. For example, each lateral group of spikes may include one or more first or longitudinally forward or front spike, a second or longitudinally middle spike, and a third or longitudinally rear spike, such that the longitudinally middle spike is disposed longitudinally between the forward spike and the rear spike, and forward of the central lateral axis. The spikes of each lateral group may be constructed substantially identically and symmetrically. In some embodiments, the spikes in each group are laterally spaced from each other relative to the longitudinal axis. For example, the first or longitudinally forward spike may be disposed laterally between the middle and rear spikes. Alternatively, the first forward spike may be disposed closer or further away from longitudinal axis 430 than the middle and rear spikes.

In some embodiments, the spikes in each group are laterally spaced from each other relative to the longitudinal axis. In some embodiments, the spikes in each group are longitudinally spaced from each other. In other embodiments, the spikes in each group are aligned with each other in the longitudinal direction.

Each spike may have any suitable shape, such as triangular, square, rectangular, oval, cylindrical or the like. In one embodiment, spikes have a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus define a transverse axis that extends transversely between the outer tip and the bone facing. The spikes may define recesses therein that extend from a portion of spikes between the base and the tip and into, but not through, endplates 210, 220. A more complete description of suitable spikes that may be incorporated into implant 200 can be found in commonly assigned U.S. Pat. No. 8,858,636, the complete disclosure of which is incorporated herein by reference for all purposes.

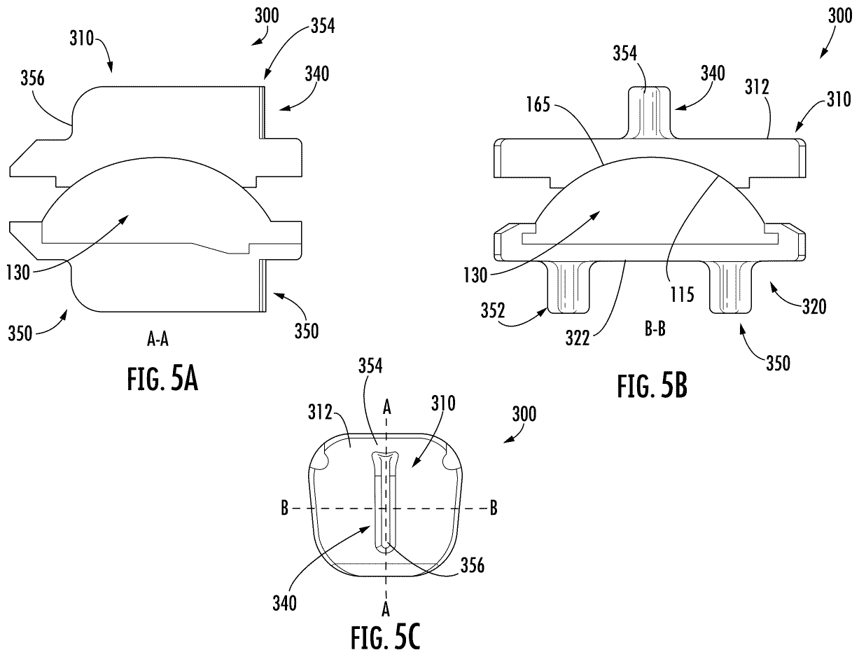

Referring now to FIGS. 5A-5C, a spinal implant 300 comprises an upper or superior endplate 310, a lower or inferior endplate 320 and a core 130. Superior endplate 310 rides upon the core 130 and is operable to rotate relative to core 130, including torsional rotation about a vertical axis, flexion/extension rotation in the posterior/anterior directions and lateral bending rotation. Similar to implants 100 and 200, core 130 includes a substantially spherical dome 160 having a convex upper surface 165 that contacts an internal concave surface 115 of upper endplate 310 and an inlay 170 that is slidably disposed within longitudinal recesses or slots in lower endplate 320.

Convex upper surface 165 of dome 160 has a surface area that is larger than the surface area of concave inner surface 115 of upper endplate 310. Accordingly, dome 160 is “oversized” relative to concave inner surface 115, which increases the spacing between upper and lower endplates 310, 320 in the anatomically neutral position shown, for example, in FIG. 5A. Also similar to the previous embodiments, the oversized dome configuration also provides an increased range of motion of implant 300. In particular, since upper endplate 310 is spaced further away from lower endplate 320 and dome 160 has a larger surface area than inner concave surface 115 of upper endplate 310, upper endplate 310 may rotate relative to lower endplate 320 over a greater total angle about axes A-A and B-B (shown in FIG. 1A).

In one embodiment, the upper bone contacting surface 312 of upper endplate 310 is substantially flat and the side surfaces of upper endplate 310 are substantially perpendicular to the upper surface and include rounded or beveled corners. Similarly, the lower bone contacting 322 surface of lower endplate 320 may be substantially flat and the side surfaces of lower endplate 320 are substantially perpendicular to the lower surface and include rounded or beveled corners. In some embodiments, upper and/or lower endplates 310, 320 have recesses or notches (not shown) carved into one or more of the corners.

In other embodiments, bone contacting surface 312 and/or surface 322 may have a substantially domed shape such that part of, or the entire, contact surface is curved. In some embodiments, the contact surfaces may be generally convex from an anterior edge of each surface to the posterior edge of each surface. Similarly, the contact surfaces may be generally convex from a first lateral edge of the surface to a second lateral edge opposite the first lateral edge.

Implant 300 further includes a first keel 340 extending from the upper surface 312 of upper endplate 310 and second and third keels 350, 352 extending from the lower bone contacting surface 322 of lower endplate 320. First keel 340 generally extends centrally along the longitudinal axis of implant 300 and second and third keels 350, 352 generally extend parallel to the longitudinal axis, but are laterally offset from this axis. Keels 350, 352 are preferably spaced laterally an equal distance from a longitudinal axis of the implant 300.

Keels 340, 350, 352 each include an anterior or trailing end 354 that flares laterally outward to anchor this end of the keels in their cutouts in adjacent vertebrae. The posterior or leading end 356 of the keels 340, 350, 352 are rounded or beveled to facilitate insertion of the keels into cutouts formed in the adjacent vertebrae. Keels 340, 350, 352 each extend along the bone contacting surfaces such that a space exists between the anterior and posterior surfaces of the bone contacting surfaces of endplates 310, 320 and the keels. The sides surfaces of keels 340, 350, 352 are substantially perpendicular to the bone contacting surfaces of endplates 310, 320, but, in some embodiments, may also taper inward in the directions away from these bone contacting surfaces such that the upper surface of keels 340, 350, 352 has a smaller width than the base. The upper surfaces of keels 340, 350, 352 are preferably rounded or beveled to create a smooth curved transition from the side surfaces to the top surfaces. A more complete description of suitable keels that may be incorporated into implant 300 can be found in commonly assigned, U.S. Pat. No. 8,998,990, the complete disclosure of which is incorporated herein by reference for all purposes.

Referring now to FIGS. 6A and 6B, another embodiment of a spinal implant 100a will now be described. Note that FIGS. 6A and 6B only illustrate a portion of implant 100a, namely, a lower portion or surface of the upper endplate 110a and a portion of the spherical dome 130a. Implant 100a may have any of the configurations described above, including any combination of the fixation elements described herein (e.g., keel(s) and spike(s)). In some embodiments, implant 100a will include the “oversized” dome described above. In other embodiments, the dome will not be oversized such that the ratio of the surface area of convex surface 165a and the surface area of concave surface 115a may be about 1, greater than 1, or less than 1.

In this embodiment, the lower surface 115a of upper endplate 110a has a larger radius of curvature than the upper surface 165a of spherical dome 130a (as shown in FIG. 6A). In some embodiments, lower surface 115a is the concave inner surface of upper endplate 110a. In other embodiments, lower surface 115 is the concave inner surface of an internal cup coupled to upper endplate (not shown). Surfaces 115a and 165a each generally define an arc of a circle having a certain radius. The radius of the circle defined by surface 115a is greater than the radius of the circle defined by surface 165a. Thus, surface 115a is “flatter” than surface 165a such that small gaps 180, 182 may be present between these two surfaces at either end. After implantation, the weight of the patient's body creates a load that, over time, creates a deformity in certain components of the endplate, such as the spherical dome 130a. This causes dome 130a to flatten out such the radius of curvature of surface 115a increases (see FIG. 6D).

In an exemplary embodiment, the radius of curvature of surfaces 115a and 165a are selected such that, these radii are substantially equal to each other under the load created by the weight of the patient's body (as shown in FIG. 6D). This load may depend on a variety of factors, such as the location of implant 100a in the patient's spine (i.e., lumbar implants may be subject to greater loads than cervical implants), the overall weight of the patient, and other anatomical factors.

For example, a certain percentage of the overall body weight is applied to the implant depending on its location within the spine. For a lumbar implant, approximately 50% of the overall body weight of an individual may be applied to the implant when it is implanted into an intervertebral space in the lumbar region of the spine. For a cervical implant, this percentage will be substantially less, and may be, for example, less than 25% of the overall body weight of the patient. In an exemplary embodiment, if the patient's body weight is below about 50 kilograms, the radius of the circles defined by surfaces 115a and 165a may be substantially the same (or the radius of surface 115a may be only marginally greater than surface 165a, e.g., less than 10 microns) because this overall body weight may produce only slight or no deformation of dome 130a. If the patient's body weight is between about 50 kilograms to about 100 kilograms, the radius of surface 115a may be increased relative to surface 165a by about 19 microns to about 50 microns to accommodate this load. If the patient's overall body weight is greater than about 100 kilograms, the radius of surface 115a may be increased relative to surface 165a by about 40 to about 100 microns to accommodate this load.

In some embodiments, the size of gaps 180, 182 and the relative radii of surfaces 115a and 165a may be selected such that translation and rotation of upper endplate 110a relative to dome 130a occurs primarily in response to flexion and extension of the patient's spine, rather than independently at rest. For example, the radius of curvature of surface 115a may be slightly larger than surface 165a under no load, leaving minimal gaps that prevent free translation or rotation. During flexion or extension, physiological loads cause the endplate 110a to move relative to dome 130a, allowing controlled translation and rotation while maintaining contact at one side and opening a gap at the opposite side.

This controlled movement may be tuned by adjusting the difference in radii of curvature between surfaces 115a and 165a, the size and geometry of gaps 180, 182, and the stiffness of the materials forming dome 130a and endplate 110a. The gaps are selected to permit relative motion primarily during flexion/extension rather than independently, thereby mimicking natural spinal articulation.

FIG. 6B illustrates translation of the upper endplate 110a relative to the spherical dome 130a (i.e., to the right in FIG. 6B although it will be recognized that endplate 110a could also translate to the left). As shown, gaps 180, 182 allow upper endplate 110a to translate relative to dome 130a. In particular, as endplate 110a translates to the right, gap 180 becomes closed such that surface 115a contacts surface 165a at this side of dome 130a. Gap 182 becomes larger as surface 115a moves away from surface 165a at this side of dome 130a. This allows upper endplate 110a to translate relative to dome 130a during flexion/extension of the patient's spine.

FIG. 6C illustrates rotation of the upper endplate 110a relative to the spherical dome 130a (i.e., to the right in FIG. 6C although it will be recognized that endplate 110a could also rotate to the left). As shown, gaps 180, 182 allow upper endplate 110a to rotate relative to dome 130a. In particular, as endplate 110a rotates to the right, gap 180 becomes closed such that surface 115a contacts surface 165a at this side of dome 130a. Gap 182 becomes larger as surface 115a moves away from surface 165a at this side of dome 130a. This allows upper endplate 110a to rotate relative to dome 130a during flexion/extension of the patient's spine.

Of course, it will be recognized that upper endplate 110a may both translate and rotate relative to dome 130a. In this configuration, gaps 180, 182 would accommodate both of these movements.

Referring now to FIGS. 7A and 7B, another embodiment of a spinal implant 400 will now be described. As in previous embodiments, spinal implant 400 comprises an upper or superior endplate 410, a lower or inferior endplate 440 and a core 430. Superior endplate 410 rides upon the core 430 and is operable to rotate relative to core 430, including torsional rotation about a vertical axis, flexion/extension rotation in the posterior/anterior directions and lateral bending rotation. Core 430 includes a substantially spherical dome 460 having a convex upper surface 465 that contacts an internal concave surface (not shown) of upper endplate 410 and an inlay 470 that is slidably disposed within longitudinal recesses or slots in lower endplate 420.

As shown in FIG. 7B, spherical dome 460 is tilted at an angle relative to inlay 470 and, therefore, dome 460 is generally tilted at an angle relative to an inner surface 480 of lower endplate 420. In one embodiment, dome 460 is titled towards the posterior or leading direction of implant 400. This creates an implant with a built-in lordotic angle that may accommodate a degree of posterior curvature within a curved portion of the patient's spine (i.e., cervical or lumbar lordosis).

Inner surface 480 of lower endplate 420 generally extends along a plane substantially parallel to a longitudinal axis of the implant. Dome 460 defines a center of curvature and an axis 482 extending from the center of curvature to an apex of the outer convex surface 465. This axis extends at an acute angle relative to inner surface 480 of lower endplate 420 and, in some embodiments, an upper surface 484 of inlay 170. The angle may be about 75 degrees to about 89 degrees, or about 85 degrees to about 88 degrees. In an exemplary embodiment, the angle is about 87 degrees.

Dome 460 further defines a second surface 492 extending between convex surface 465 and inner surface 484 of inlay 470 (see FIG. 6B). Second surface 492 is a result of the tilted convex surface 465 (i.e., convex surface 465 has a first or trailing end 491 that is raised upwards from its leading end 490). Second surface 492 may be transverse to, or substantially perpendicular to, upper surface 480 of lower endplate 420.

In some embodiments, dome 460 is eccentric or not centered relative to upper and lower endplates 410, 420. Endplates 410, 420 have a leading or posterior end 450, a trailing or anterior end 452, and a midline axis 454 extending longitudinally between posterior and anterior ends 450, 452. As used herein, the term “eccentric” (and grammatical variants thereof) refers to a structure, feature, or component that is offset, displaced, or positioned away from a central axis, centerline, geometric center, or axis of rotation of another structure. An eccentric element may be offset by any non-zero distance and in any radial or lateral direction. In this embodiment, dome 460 is shifted in the posterior direction such that dome 460 is positioned closer to posterior end 450 than anterior end 452. In particular, spherical dome 460 has an apex 462 that defines an axis 482 extending from the center of curvature to an apex of the outer convex surface 465. The posterior shift of dome 460 moves the center of rotation (COR) of implant 400 in a posterior direction relative to the midline axis 454. This posteriorly-shifted COR effectively offsets the tilt of dome 460, helping to align the implant's COR with the natural anatomical COR of the vertebral segment and improving physiological motion of the implant.

In addition, in certain anatomies, the orientation of the inferior and superior facet processes may be more vertical than normal, i.e., the facet processes are oriented at an angle greater than 30-45 degrees relative to the horizontal axis in the sagittal plane. In this configuration, the center of rotation of the functional spinal unit is translated more posteriorly toward the posterior border of the vertebral body. In addition, the radius of the rotation amidst flexion or extension can be smaller than normal. A more vertical orientation of the facets leads to less translation at maximum flexion. This more vertical orientation of the facets induces a more posterior position of the center of rotation in the sagittal plane for flexion extension, and by consequence a smaller radius.

In an exemplary embodiment, dome axis 482 is spaced posteriorly from midline axis 454 by a distance of about 1 mm to about 10 mm, or about 2 mm to about 4 mm for cervical implants, and about 2 mm to about 8 mm for lumbar implants. Convex upper surface 465 of dome 460 generally has a posterior surface area located on the posterior side of midline axis 454 and an anterior surface area located on the anterior side of midline axis 454. The posterior surface area is greater than the anterior surface area. In an exemplary embodiment, the posterior surface area of surface 165 is about 45% to about 80%, or about 50% to about 75%, or about 55% to about 70% of the anterior surface area of surface 165.

Implant 400 may include any configuration of keels and/or spikes described above. In the representative embodiment, implant 400 includes a first keel 440 extending from the upper bone contacting surface of upper endplate 410 and a second keel 550 extending from the lower bone contacting surface of lower endplate 420. Keels 440, 450 are centrally located on endplates 410, 420 such that they generally extend along a longitudinal axis of the endplates. Keels 440, 450 may have any of the configurations described above.

Upper and lower endplates 410, 420 may have substantially flat bone contacting surfaces, or these surfaces may have a substantial dome shape as described above.

Referring now to FIGS. 8A and 8B, another embodiment of a spinal implant 500 will now be described. As in previous embodiments, spinal implant 500 comprises an upper or superior endplate 510, a lower or inferior endplate 520 and a core 530. Superior endplate 510 rides upon the core 530 and is operable to rotate relative to core 530, including torsional rotation about a vertical axis, flexion/extension rotation in the posterior/anterior directions and lateral bending rotation. Core 530 includes a substantially spherical dome 560 having a convex upper surface 565 that contacts an internal concave surface (not shown) of upper endplate 510 and an inlay 570 that is slidably disposed within longitudinal recesses or slots in lower endplate 520.

In this embodiment, upper endplate 510 includes an internal cup 517 that extends downwardly from upper endplate 510 and has an internal concave surface (not shown) that contacts convex surface 565 of dome 560. Internal cup 517 generally defines a conical shape and has a substantially annular base surface 516 facing lower endplate 510. The base surface 516 extends around a portion of dome 560 and is substantially parallel to an upper surface 580 of lower endplate 520 in an anatomically neutral position (shown in FIG. 8A).

In various embodiments, the upper endplate 510 is movable between a first anatomically neutral position to a second rotated position. Upper endplate 510 comprises a lower surface 556 that substantially surrounds internal cup 517 such that cup 517 extends downwardly from lower surface 556. Lower surface 556 of upper endplate 510 is generally disposed at an acute angle relative to base surface 516 of internal cup 517 such that upper endplate 510 is “tilted” relative to lower endplate 520 in the anatomically neutral position shown in FIG. 8A. In an exemplary embodiment, lower surface 556 is titled at an acute angle relative to base surface 516 of internal cup 517. This acute angle may be about 1 degree to about 15 degrees, or about 5 degrees to about 2 degrees, or about 3 degrees.

In this configuration, upper endplate 510 includes a built-in lordotic angle (rather than spherical dome 560 as in the previous embodiment) that may accommodate or compensate for a natural degree of posterior curvature within a curved portion of the patient's spine (i.e., cervical or lumbar lordosis). In addition, in a disc space that is in extension (i.e., space lordosis), the angled superior endplate absorbs at least a part of the requirement for a lordosis angle and increases the range of motion in extension instead of using this degree of angle in static compensation, as discussed in more detail below.

In this configuration, internal cup 517 has a first or leading end 514 that is thinner than a second or trailing end 518. In other words, the length or distance of the posterior surface of leading end 514 that extends downwardly from lower surface 556 of upper endplate is less than the length or distance of the anterior surface of trailing end 518. In an exemplary embodiment, the length or thickness of the first leading end 514 is greater than the length or thickness of the second trailing end 518.

Implant 500 may include any configuration of keels and/or spikes described above. In the representative embodiment, implant 500 includes a first keel 540 extending from upper bone contacting surface 512 of upper endplate 510 and a second keel 550 extending from lower bone contacting surface 522 of lower endplate 520. Keels 540, 550 are centrally located on endplates 510, 520 such that they generally extend along a longitudinal axis of the endplates. Keels 540, 550 may have any of the configurations described above.

Upper and lower endplates 510, 520 may have substantially flat bone contacting surfaces, or these surfaces may have a substantial dome shape as described above.

FIGS. 8C and 8D illustrate an intervertebral space that is in extension (i.e., space lordosis). Thus, the upper vertebrae is angled relative to the lower vertebrae such that the upper endplate of the implant resides at an angle relative to the lower endplate in the anatomically neutral position shown in these figures. FIG. 8C illustrates a conventional implant 590 that includes an upper endplate 592 and a lower endplate 594. Upper endplate 592 includes an internal cup 596 that articulates with a core member 598 between upper and lower endplates 592, 594. The internal cup 596 has a lower base surface 599 that is generally parallel to a lower surface 593 of endplate 592.

When implant 590 is implanted into an intervertebral space with space lordosis, the range of motion is reduced, particularly in extension because the upper endplate 592 is already rotated or tilted relative to the lower endplate 594 in the anatomically neutral position. This reduces the amount of anterior rotation that the upper endplate can make within the space.

FIG. 8D illustrates the spinal implant 500 of FIGS. 8A and 8B implanted in an intervertebral space that is in extension (i.e., space lordosis). As shown, internal cup 517 has a base surface 516 that is disposed at an angle relative to the lower surface 556 of upper endplate 510. Thus, in the anatomically neutral position shown, while upper endplate 510 is tilted relative to lower endplate 520, internal cup 517 is positioned such that the base surface 516 that contacts the dome 530 is substantially parallel to upper surface 580 of lower endplate 520. This allows internal cup 517 to have a larger range of motion than conventional implants, particularly in the anterior/posterior direction or in extension.

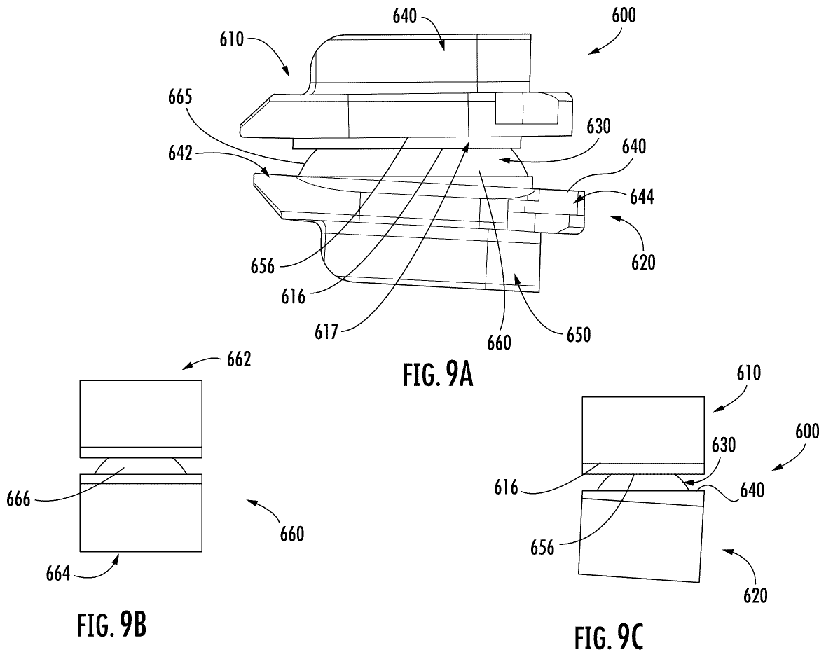

Referring now to FIG. 9A, another embodiment of a spinal implant 600 will now be described. As in previous embodiments, spinal implant 600 comprises an upper or superior endplate 610, a lower or inferior endplate 620 and a core 630. Superior endplate 610 rides upon the core 630 and is operable to rotate relative to core 630, including torsional rotation about a vertical axis, flexion/extension rotation in the posterior/anterior directions and lateral bending rotation. Core 630 includes a substantially spherical dome 660 having a convex upper surface 665 that contacts an internal concave surface (not shown) of upper endplate 610.

In this embodiment, lower endplate 620 is tilted relative to upper endplate 610 and core 630. Thus, lower endplate 620 comprises an upper surface 640 that is titled at an angle relative to lower surface 656 of upper endplate 610 and base surface 616 of the internal cup 617 of upper endplate 610 in the anatomically neutral position shown in FIG. 9A. In particular, dome 660 has a generally spherical shape and thus defines an apex (not shown) in contact with the upper portion of the concave upper surface of cup 617. Dome 660 defines a radius extending from the apex to a theoretical center of the sphere defined by dome 660. This radius is generally perpendicular to lower surface 656 of upper endplate 610 and base surface 616 of the internal cup 617. Upper surface 640 of lower endplate 620, however, is disposed at an acute angle relative to this radius (i.e., not perpendicular). In an exemplary embodiment, this acute angle may be about 1 degree to about 15 degrees, or about 5 degrees to about 2 degrees, or about 3 degrees.

In some embodiments, lower endplate 520 may have a first or leading end 642 that is thinner than a second or trailing end 644. In other words, the length or distance of the posterior surface of leading end 642 is less than the length or distance of the anterior surface of trailing end 644.

Implant 600 may include any configuration of keels and/or spikes described above. In the representative embodiment, implant 600 includes a first keel 640 extending from upper bone contacting surface 612 of upper endplate 610 and a second keel 650 extending from lower bone contacting surface 622 of lower endplate 620. Keels 640, 650 are centrally located on endplates 610, 620 such that they generally extend along a longitudinal axis of the endplates. Keels 640, 650 may have any of the configurations described above.

Upper and lower endplates 610, 620 may have substantially flat bone contacting surfaces, or these surfaces may have a substantial dome shape as described above.

FIGS. 9B and 9C illustrate an intervertebral space that includes a trapezoidal shape with an anterior portion higher than a posterior portion. In this anatomy, there is a reduced translation in flexion for a conventional implant within this intervertebral space. FIG. 9B illustrates a conventional implant 660 having upper and lower endplates 662, 664 and an articulating core member 666 therebetween. As shown, in the anatomically neutral position, the upper surface of lower endplate 664 is generally parallel to the lower surface of upper endplate 662 and to the base surface of the internal cup that extends around core member 666. This reduces the amount of translation in flexion that the upper endplate can make within the space.

FIG. 9C illustrates the implant 600 of FIG. 9A in an intervertebral space that includes a trapezoidal shape with an anterior portion higher than a posterior portion. As discussed previously, lower endplate 620 is tilted relative to upper endplate 610 and core 630. Thus, lower endplate 620 comprises an upper surface 640 that is titled at an angle relative to lower surface 656 of upper endplate 610 and base surface 616 of the internal cup 617 of upper endplate 610. This allows for increased translation in flexion in the intervertebral space.

Other embodiments will be apparent to those skilled in the art from consideration of the specification and practice of the embodiments disclosed herein. It is intended that the specification and examples be considered as exemplary only, with a true scope and spirit of the embodiment being indicated by the following claims.

For example, in a first aspect, a first embodiment is an implant for insertion into an intervertebral disc space between two adjacent vertebrae. The implant comprises a first component having an outer surface for engaging one of the adjacent vertebrae and a concave surface opposite the outer surface, a second component having an outer surface for engaging the other of the adjacent vertebrae and an inner surface opposite the outer surface and an articulating core member residing between the first and second components. The articulating core member comprises a convex surface in contact with the concave surface of the first component, wherein a surface area of the convex surface is greater than a surface area of the concave surface.

A second embodiment is the first embodiment, wherein a ratio between the surface area of the convex surface and the surface area of the concave surface is about 1.5 to about 1.002.

A third embodiment is any combination of the above embodiments, wherein the ratio is about 1.4 to about 1.1.

A 4^{th} embodiment is any combination of the above embodiments, wherein the ratio is about 1.3 to about 1.2.

A 5^{th} embodiment is any combination of the above embodiments, wherein a ratio between a spherical radius of the convex surface and a spherical radius of the concave surface is about 1.4 to about 1.1.

A 6^{th} embodiment is any combination of the above embodiments, wherein the first component is configured to articulate relative to the second component around an axis from a first position, wherein the first and second components are substantially parallel, to a second position, wherein the first component is disposed at an angle of at least about 7 degrees relative to the second component.

A 7^{th} embodiment is any combination of the above embodiments, wherein the angle is at least about 11.5 degrees.

An 8^{th} embodiment is any combination of the above embodiments, wherein the axis is substantially perpendicular to a longitudinal axis of the implant.

A 9^{th} embodiment is any combination of the above embodiments, wherein the convex surface defines an arc from one end of the convex surface to an opposite end of the convex surface and the concave surface defines an arc from one end of the concave surface to an opposite end of the concave surface, wherein a length of the convex surface arc is greater than a length of the concave surface arc.

A 10^{th} embodiment is any combination of the above embodiments, wherein a ratio of the length of the convex surface arc to the length of the concave surface arc is about 1.5 to about 1.002.

An 11^{th} embodiment is any combination of the above embodiments, wherein the ratio is about 1.4 to about 1.1.

A 12^{th} embodiment is any combination of the above embodiments, wherein the ratio is about 1.3 to about 1.2.

A 13^{th} embodiment is any combination of the above embodiments, wherein the concave surface arc of the first component has a first radius and the convex surface arc of the core member has a second radius less than the first radius.

A 14^{th} embodiment is any combination of the above embodiments, wherein the articulating core member comprises a semi-spherical member and an inlay, wherein the inlay is coupled to the second component and comprises a recess for receiving the semi-spherical member.

A 15^{th} embodiment is any combination of the above embodiments, wherein the first component is an upper endplate and comprises a fixation element extending from the outer surface and wherein the second component is a lower endplate and comprises a fixation element extending from the outer surface.

A 16^{th} embodiment is any combination of the above embodiments, wherein the fixation element on the upper and lower endplates comprises one or more spikes.

A 17^{th} embodiment is any combination of the above embodiments, wherein the fixation element on the upper and lower endplates comprises one or more keels.

In another aspect, a first embodiment is an implant for insertion into an intervertebral disc space between two adjacent vertebrae. The implant comprises a first component having an outer surface for engaging one of the adjacent vertebrae and a concave surface opposite the outer surface, the concave surface having a first radius of curvature, a second component having an outer surface for engaging the other of the adjacent vertebrae and an inner surface opposite the outer surface and an articulating core member residing between the first and second components. The articulating core member comprising a convex surface in contact with the concave surface of the first component, the convex surface having a second radius of curvature less than the first radius of curvature.

A second embodiment is the first embodiment, wherein the first component is an upper endplate and the second component is a lower endplate.

A third embodiment is any combination of the above embodiments, wherein the convex surface defines an arc from one end of the convex surface to an opposite end of the convex surface and the concave surface defines an arc from one end of the concave surface to an opposite end of the concave surface, wherein the concave surface arc has a first radius and the convex surface arc has a second radius less than the first radius.

A 4^{th} embodiment is any combination of the above embodiments, wherein the intervertebral disc space is a lumbar intervertebral disc space.

A 5^{th} embodiment is any combination of the above embodiments, wherein the first radius is about 10 microns to about 100 microns greater than the second radius.

A 6^{th} embodiment is any combination of the above embodiments, wherein the first radius is about 19 to about 50 microns greater than the second radius.

A 7^{th} embodiment is any combination of the above embodiments, wherein the first radius is about 40 microns about 100 microns greater than the second radius.

An 8^{th} embodiment is any combination of the above embodiments, wherein the first component is an upper endplate and comprises a fixation element extending from the outer surface and wherein the second component is a lower endplate and comprises a fixation element extending from the outer surface.

In another aspect, a first embodiment is an implant for insertion into an intervertebral disc space between two adjacent vertebrae. The implant comprises a first component having an outer surface for engaging one of the adjacent vertebrae and a concave surface opposite the outer surface, a second component having an outer surface for engaging the other of the adjacent vertebrae and an inner surface opposite the outer surface and an articulating core member residing between the first and second components. The articulating core member comprises a semi-spherical protrusion with a convex surface in contact with the concave surface of the first component, wherein the semi-spherical protrusion is tilted at an angle relative to the inner surface of the second component.

A second embodiment is the first embodiment, wherein the inner surface of the second component extends along a plane substantially parallel to a longitudinal axis of the implant.

A third embodiment is any combination of the above embodiments, wherein the semi-spherical protrusion defines a center of curvature and an axis extending from the center of curvature to an outer surface of the protrusion, wherein said axis extends at an acute angle relative to the inner surface of the second component.

A 4^{th} embodiment is any combination of the above embodiments, wherein the acute angle is about 75 degrees to about 89 degrees.

A 5^{th} embodiment is any combination of the above embodiments, wherein the acute angle is about 85 degrees to about 88 degrees.

A 6^{th} embodiment is any combination of the above embodiments, wherein the acute angle is about 87 degrees.

A 7^{th} embodiment is any combination of the above embodiments, wherein the semi-spherical protrusion comprises a first semi-spherical surface and a second surface extending from the semi-spherical surface to the inner surface of the second component.

An 8^{th} embodiment is any combination of the above embodiments, wherein the second surface is substantially perpendicular to the inner surface of the second component.

A 9^{th} embodiment is any combination of the above embodiments, wherein at least a portion of the first semi-spherical surface contacts at least a portion of the inner surface of the second component.

A 10^{th} embodiment is any combination of the above embodiments, wherein the first component is an upper endplate and comprises a fixation element extending from the outer surface and wherein the second component is a lower endplate and comprises a fixation element extending from the outer surface.

An 11^{th} embodiment is any combination of the above embodiments, wherein the fixation element on the upper and lower endplates comprises one or more spikes.

A 12^{th} embodiment is any combination of the above embodiments, wherein the fixation element on the upper and lower endplates comprises one or more keels.

A 13^{th} embodiment is any combination of the above embodiments, wherein the articulating core member is eccentric to the first and/or the second component.

A 14^{th} embodiment is any combination of the above embodiments, wherein the second component has a posterior end and an anterior end and a midline axis extending through the second component equally distant between the anterior and posterior ends and wherein the core member comprises an apex posterior to the midline axis of the second component.

A 15^{th} embodiment is any combination of the above embodiments, wherein the apex is spaced about 2 mm to about 10 mm from the midline axis of the second component.

A 16^{th} embodiment is any combination of the above embodiments, wherein the convex surface defines a posterior surface area posterior to the midline axis of the second component and an anterior surface area anterior to the midline axis of the second component, wherein the posterior surface area is greater than the anterior surface area.

A 17^{th} embodiment is any combination of the above embodiments, wherein the posterior surface area is about 45% to about 80% of the anterior surface area.

In another aspect, a first embodiment is an implant for insertion into an intervertebral disc space between two adjacent vertebrae. The implant comprises a first component having an outer surface for engaging one of the adjacent vertebrae and an internal cup having a concave surface with a substantially annular distal base surface, the first component further comprising an inner surface substantially surrounding the concave surface opposite the outer surface, a second component having an outer surface for engaging the other of the adjacent vertebrae and an inner surface opposite the outer surface and an articulating core member residing between the first and second components, the articulating core member comprising a convex surface in contact with the concave surface of the internal cup of the first component. The distal base surface of the internal cup is disposed at a transverse angle relative to the inner surface of the first component.

A second embodiment is the first embodiment, wherein the first component is an upper endplate and the second component is a lower endplate and wherein at least a portion of the internal cup is disposed between the inner surfaces of the upper and lower endplates.

A third embodiment is any combination of the above embodiments, wherein the internal cup defines a first side surface and a second side surface extending from the inner surface of the upper endplate, wherein a length of the first side surface is greater than a length of the second side surface.

A 4^{th} embodiment is any combination of the above embodiments, wherein the first side surface is an anterior side surface and the second side surface is a posterior side surface.

A 5^{th} embodiment is any combination of the above embodiments, wherein the upper endplate is movable between a first anatomically neutral position to a second rotated position, wherein, in the first position, the distal base surface of the internal cup is substantially parallel to the inner surface of the lower endplate the inner surface of the upper endplate is disposed at a transverse angle relative to the inner surface of the lower endplate.

A 6^{th} embodiment is any combination of the above embodiments, wherein the transverse angle is about 1 degree to about 15 degrees.

A 7^{th} embodiment is any combination of the above embodiments, wherein the transverse angle is about 5 degrees to about 2 degrees.

An 8^{th} embodiment is any combination of the above embodiments, wherein the transverse angle is about 3 degrees.

A 9^{th} embodiment is any combination of the above embodiments, wherein the first component comprises a fixation element extending from the outer surface and wherein the second component comprises a fixation element extending from the outer surface.

A 10^{th} embodiment is any combination of the above embodiments, wherein the fixation element on the upper and lower endplates comprises one or more spikes.

An 11^{th} embodiment is any combination of the above embodiments, wherein the fixation element on the upper and lower endplates comprises one or more keels.

In another aspect, a first embodiment is an implant for insertion into an intervertebral disc space between two adjacent vertebrae. The implant comprises a first component having an outer surface for engaging one of the adjacent vertebrae and a concave surface opposite the outer surface, the first component further comprising an inner surface substantially surrounding the concave surface, a second component having an outer surface for engaging the other of the adjacent vertebrae and an inner surface opposite the outer surface and an articulating core member residing between the first and second components, the articulating core member comprising a convex surface in contact with the concave surface of the first component. The inner surface of the second component is disposed at a transverse angle relative to the inner surface of the first component.

A second embodiment is the first embodiment, wherein the first component is an upper endplate and the second component is a lower endplate.

A third embodiment is any combination of the above embodiments, wherein the articulating core member comprises an apex and a radius extending downwardly from the apex, and wherein the inner surface of the second component is disposed at a transverse angle relative to the radius.

A 4^{th} embodiment is any combination of the above embodiments, wherein the transverse angle is about 75 degrees to about 89 degrees.

A 5^{th} embodiment is any combination of the above embodiments, wherein the transverse angle is about 85 degrees to about 88 degrees.

A 6^{th} embodiment is any combination of the above embodiments, wherein the transverse angle is about 87 degrees.

A 7^{th} embodiment is any combination of the above embodiments, wherein the upper endplate is movable between a first anatomically neutral position to a second rotated position, wherein, in the first position, the inner surface of the upper endplate is substantially parallel to the radius and the inner surface of the lower endplate is disposed at a transverse angle relative to the radius.

An 8^{th} embodiment is any combination of the above embodiments, wherein the first component comprises a fixation element extending from the outer surface and wherein the second component comprises a fixation element extending from the outer surface.

A 9^{th} embodiment is any combination of the above embodiments, wherein the fixation element on the upper and lower endplates comprises one or more spikes.

A 10^{th} embodiment is any combination of the above embodiments, wherein the fixation element on the upper and lower endplates comprises one or more keels.

1. An implant for insertion into an intervertebral disc space between two adjacent vertebrae, the implant comprising:
a first component having an outer surface for engaging one of the adjacent vertebrae and a concave surface opposite the outer surface;
a second component having an outer surface for engaging the other of the adjacent vertebrae and an inner surface opposite the outer surface; and
an articulating core member residing between the first and second components, the articulating core member comprising a convex surface in contact with the concave surface of the first component, wherein a surface area of the convex surface is greater than a surface area of the concave surface.

2. The implant of claim 1, wherein a ratio between the surface area of the convex surface and the surface area of the concave surface is about 1.5 to about 1.002.

3. The implant of claim 1, wherein a ratio between a spherical radius of the convex surface and a spherical radius of the concave surface is about 1.4 to about 1.1.

4. The implant of claim 1, wherein the first component is configured to articulate relative to the second component around an axis from a first position, wherein the first and second components are substantially parallel, to a second position, wherein the first component is disposed at an angle of at least about 7 degrees relative to the second component.

5. The implant of claim 1, wherein the convex surface defines an arc from one end of the convex surface to an opposite end of the convex surface and the concave surface defines an arc from one end of the concave surface to an opposite end of the concave surface, wherein a length of the convex surface arc is greater than a length of the concave surface arc.

6. The implant of claim 5, wherein a ratio of the length of the convex surface arc to the length of the concave surface arc is about 1.5 to about 1.002.

7. The implant of claim 5, wherein the concave surface arc of the first component has a first radius and the convex surface arc of the core member has a second radius less than the first radius.

8. An implant for insertion into an intervertebral disc space between two adjacent vertebrae, the implant comprising:
a first component having an outer surface for engaging one of the adjacent vertebrae and a concave surface opposite the outer surface, the concave surface having a first radius of curvature;
a second component having an outer surface for engaging the other of the adjacent vertebrae and an inner surface opposite the outer surface; and
an articulating core member residing between the first and second components, the articulating core member comprising a convex surface in contact with the concave surface of the first component, the convex surface having a second radius of curvature less than the first radius of curvature.

9. The implant of claim 8, wherein the convex surface defines an arc from one end of the convex surface to an opposite end of the convex surface and the concave surface defines an arc from one end of the concave surface to an opposite end of the concave surface, wherein the concave surface arc has a first radius and the convex surface arc has a second radius less than the first radius.

10. The implant of claim 8, wherein the first radius is about 10 microns to about 100 microns greater than the second radius.

11. The implant of claim 8, wherein the first radius is about 40 microns about 100 microns greater than the second radius.

12. The implant of claim 8, wherein the first component is an upper endplate and comprises a fixation element extending from the outer surface and wherein the second component is a lower endplate and comprises a fixation element extending from the outer surface.

13. An implant for insertion into an intervertebral disc space between two adjacent vertebrae, the implant comprising:
a first component having an outer surface for engaging one of the adjacent vertebrae and a concave surface opposite the outer surface;
a second component having an outer surface for engaging the other of the adjacent vertebrae and an inner surface opposite the outer surface; and
an articulating core member residing between the first and second components, the articulating core member comprising a semi-spherical protrusion with a convex surface in contact with the concave surface of the first component, wherein the semi-spherical protrusion is tilted at an angle relative to the inner surface of the second component.

14. The implant of claim 13, wherein the inner surface of the second component extends along a plane substantially parallel to a longitudinal axis of the implant.

15. The implant of claim 13, wherein the semi-spherical protrusion defines a center of curvature and an axis extending from the center of curvature to an outer surface of the protrusion, wherein said axis extends at an acute angle relative to the inner surface of the second component.

16. The implant of claim 15, wherein the acute angle is about 75 degrees to about 89 degrees.

17. The implant of claim 13, wherein the semi-spherical protrusion comprises a first semi-spherical surface and a second surface extending from the semi-spherical surface to the inner surface of the second component, wherein the second surface is substantially perpendicular to the inner surface of the second component.

18. The implant of claim 17, wherein at least a portion of the first semi-spherical surface contacts at least a portion of the inner surface of the second component.

19. The implant of claim 13, wherein the second component has a posterior end and an anterior end and a midline axis extending through the second component equally distant between the anterior and posterior ends and wherein the core member comprises an apex posterior to the midline axis of the second component, wherein the apex is spaced about 2 mm to about 10 mm from the midline axis of the second component.

20. The implant of claim 13, wherein the convex surface defines a posterior surface area posterior to the midline axis of the second component and an anterior surface area anterior to the midline axis of the second component, wherein the posterior surface area is greater than the anterior surface area, wherein the posterior surface area is about 45% to about 80% of the anterior surface area.
